(19) United States
(12) Patent Application Publication
(10) Pub. No.: US 2018/0179086 A1
(43) Pub. Date: Jun. 28, 2018

(54) STERILIZATION DEVICE

(71) Applicant: NIKKISO CO., LTD., Tokyo (JP)

(72) Inventor: Nobuhiro TORII, Hakusan-shi (JP)

(21) Appl. No.: 15/902,623

(22) Filed: Feb. 22, 2018

Related U.S. Application Data
(63) Continuation of application No. PCT/JP2016/075194, filed on Aug. 29, 2016.

(30) Foreign Application Priority Data
Sep. 3, 2015 (JP) 2015-173780

(51) Int. Cl. C02F 1/32; A61L 2/10

(52) U.S. Cl. CPC C02F 1/32; C02F 2201/002; A61L 2202/122; A61L 2/10

(57) ABSTRACT

A sterilization device includes: a flow tube that extends in a longitudinal direction; a housing that extends in the longitudinal direction along the flow tube and surrounds an outer circumference of the flow tube; and a light emitting device provided inside the housing and outside the flow tube and irradiating a fluid flowing in the flow tube with ultraviolet light. An inner surface of the housing is made of a material that reflects the ultraviolet light from the light emitting device, and a shape of a cross section of the inner surface of the housing perpendicular the longitudinal direction is non-analogous to a shape of a cross section of the flow tube perpendicular to the longitudinal direction.

### RELATED APPLICATION

This application is a Continuation of co-pending Application No. PCT/JP2016/075194 filed on Aug. 29, 2016, for which priority is claimed under 35 U.S.C. § 120; and this application claims priority of Application No. 2015-173780, filed in Japan on Sep. 3, 2015 under 35 U.S.C. § 119; the entire content of all of which are incorporated herein by reference.

### BACKGROUND OF THE INVENTION

### 1. Field of the Invention

The present invention relates to sterilization devices and, more particularly, to a device that sterilizes a fluid by irradiating the fluid with ultraviolet light.

### 2. Description of the Related Art

It is known that ultraviolet light has sterilization capability. Devices that radiate ultraviolet light are used for sterilization in medical and food processing fronts. Devices that sterilize a fluid such as water continuously by irradiating the fluid with ultraviolet light are also used. One example of such a sterilization device is a water sterilizer module in which a quartz flow tube is provided inside a housing and a plurality of ultraviolet light emitting devices are arranged along a flow path.

Provision of a quartz flow tube inside a housing requires a structure to seal between a light source unit and the flow tube, which could lead to an increase in the size and cost of the device. Usually, various equipment have already been installed in medical and food processing fronts. Considering the constraints on the space, etc., it is desired that the device be small in size.

### SUMMARY OF THE INVENTION

In this background, one illustrative purpose of the present invention is to provide a sterilization device capable of enhancing the sterilization capability with a smaller number of light emitting devices.

A sterilization device according to an embodiment comprise: a flow tube that extends in a longitudinal direction; a housing that extends in the longitudinal direction along the flow tube and surrounds an outer circumference of the flow tube; and a light emitting device provided inside the housing and outside the flow tube and irradiating a fluid flowing in the flow tube with ultraviolet light. An inner surface of the housing is made of a material that reflects the ultraviolet light from the light emitting device, and a shape of a cross section of the inner surface of the housing perpendicular the longitudinal direction is non-analogous to a shape of a cross section of the flow tube perpendicular to the longitudinal direction.

According to the embodiment, the light emitting device is provided outside the flow tube so that a member for sealing between the flow tube and the light emitting device is not necessary. Further, since the inner surface of the housing surrounding the light emitting device and the flow tube is made of a material that reflects ultraviolet light, the ultraviolet light from the light emitting device is reflected by the inner surface of the housing and radiated efficiently toward the fluid flowing in the flow tube. By configuring the shape of the inner surface of the housing and the shape of the cross section of the flow tube to be non-analogous, the intensity distribution of the ultraviolet light from the light emitting device is uniformized for irradiation of the fluid. Thus, according to the embodiment, the sterilization capability is improved by using a structure that is simple and can be easily downsized.

The shape of the cross section of the flow tube may be circular and the shape of the cross section of the inner surface of the housing is polygonal.

The shape of the cross section of the inner surface of the housing may be quadrangular.

The flow tube may be made of a resin material that transmits the ultraviolet light from the light emitting device.

The sterilization device may further comprise: a first connector connected to a first end of the flow tube; and a second connector connected to a second end of the flow tube. The housing may be sandwiched and fixed between the first connector and the second connector.

The housing may include a side wall that extends along the longitudinal direction, a first end wall formed with a first opening through which the flow tube is guided, and a second end wall formed with a second opening through which the flow tube is guided. The sterilization device may further comprise a first spacer provided between the first end wall and the first connector to surround the outer circumference of the flow tube, and a second spacer provided between the second end wall and the second connector to surround the outer circumference of the flow tube.

### BRIEF DESCRIPTION OF THE DRAWINGS

Embodiments will now be described by way of examples only, with reference to the accompanying drawings which are meant to be exemplary, not limiting and wherein like elements are numbered alike in several Figures in which:

FIG. 1 schematically shows a configuration of a sterilization device according to an embodiment; and

FIG. 2 is a sectional view schematically showing a configuration of the sterilization device of FIG. 1.

### DETAILED DESCRIPTION OF THE INVENTION

The invention will now be described by reference to the preferred embodiments. This does not intend to limit the scope of the present invention, but to exemplify the invention.

A detailed description of an embodiment to implement the present invention will be given with reference to the drawings. Like numerals are used in the description to denote like elements and the description is omitted as appropriate.

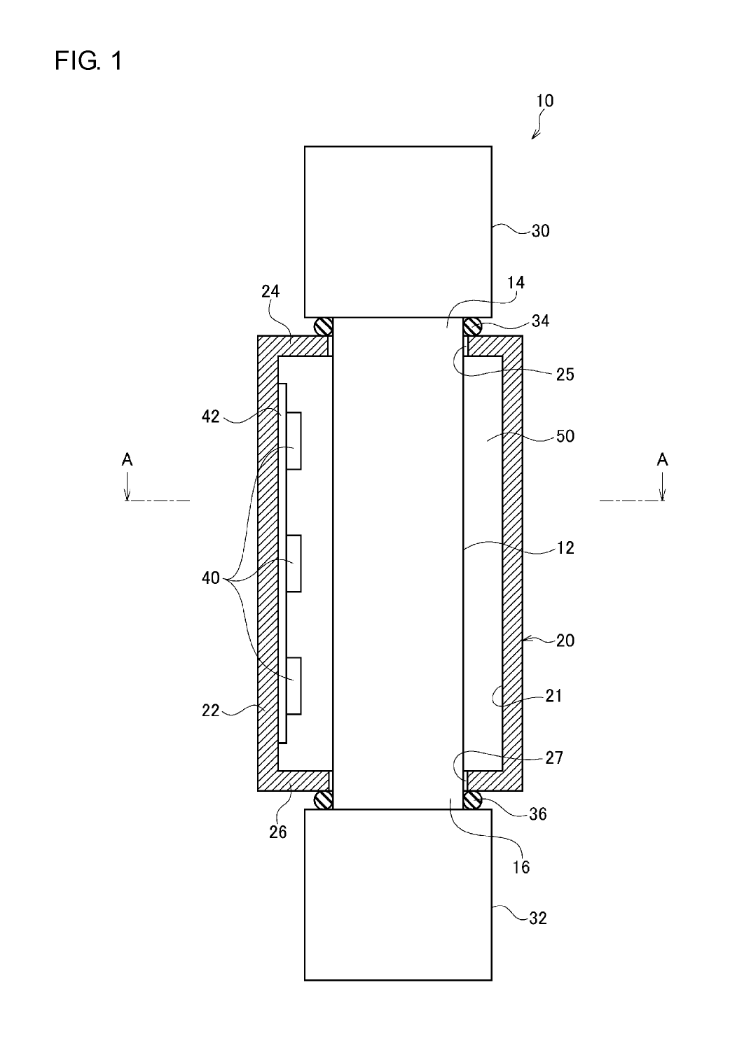

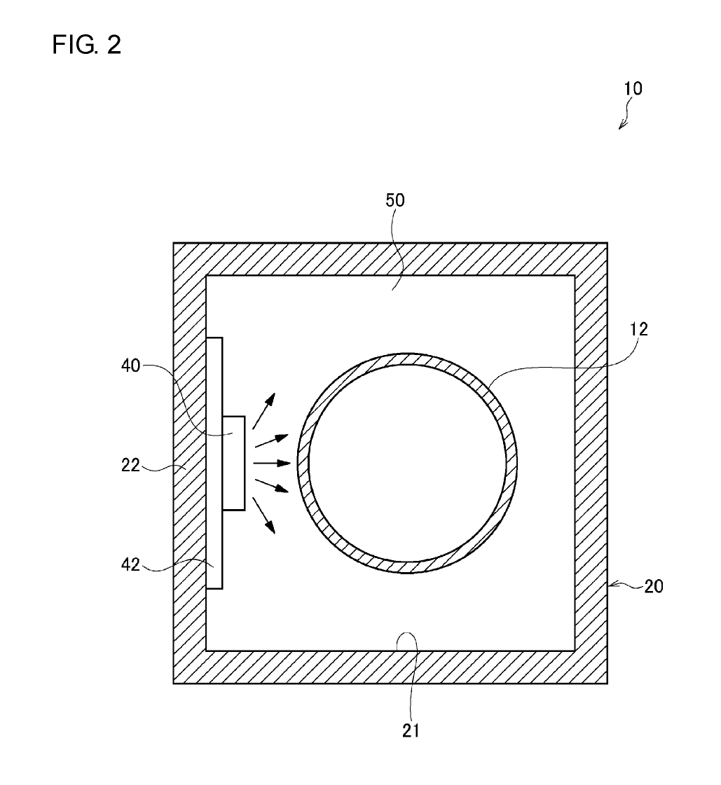

FIG. 1 schematically shows a configuration of a sterilization device 10 according to an embodiment and FIG. 2 shows an A-A cross section of FIG. 1. The sterilization device 10 is configured such that a fluid such as potable water is caused to flow into a flow tube 12 via a first connector 30, and the fluid flowing in the flow tube 12 is irradiated with ultraviolet light from a plurality of light emitting devices 40 so as to sterilize the fluid.

The sterilization device 10 includes a flow tube 12, a housing 20, a first connector 30, a second connector 32, a first spacer 34, a second spacer 36, a plurality of light emitting devices 40, and a substrate 42.

The flow tube 12 is a straight tube, the cross section thereof perpendicular to the longitudinal direction being circular. The flow tube 12 is made of a material that transmits the ultraviolet light from the light emitting devices 40. For example, the flow tube 12 is made of quartz (SiO2) or fluororesin. It is preferable that the flow tube 12 be made of fluororesin such as perfluoroalkoxyalkane (PFA) and perfluoro ethylene propylene copolymer (FEP). The flow tube 12 made of resin is configured to have a structure more resistant to impact than that of quartz glass.

The first connector 30 and the second connector 32 that are tube connectors are connected to the ends of the flow tube 12. The first connector 30 is connected to the first end 14 of the flow tube 12, and the second connector 32 is connected to the second end 16 of the flow tube 12. The first connector 30 and the second connector 32 are made of a resin material or a metallic material. For example, the first connector 30 and the second connector 32 are tube connectors of a type set inside and fixed to the flow tube 12. The first connector 30 and the second connector 32 may be tube connectors of a type fixed to the ends of the flow tube 12 in an alternative fashion.

The housing 20 has a cuboid box shape and bounds an irradiation chamber 50. The housing 20 includes a side wall 22 that extends along the longitudinal direction of the flow tube 12, a first end wall 24 formed with a first opening 25 through which the flow tube 12 is guided, and a second end wall 26 formed with a second opening 27 through which the flow tube 12 is guided.

The side wall 22 is comprised of four wall surfaces surrounding the circumference of the flow tube 12 and, as shown in FIG. 2, the cross section of the side wall 22 perpendicular to the longitudinal direction is quadrangular. The side wall 22 is formed such that the length thereof in the longitudinal direction is slightly shorter than the distance between the first connector 30 and the second connector 32. The first end wall 24 and the second end wall 26 are shaped to correspond to the cross sectional shape of the side wall 22. The first opening 25 and the second opening 27 are shaped to be slightly larger than the outer diameter of the flow tube 12.

The inner surface of the housing 20 is formed by a member having a high reflectivity for the ultraviolet light from the light emitting devices 40. For example, the inner surface is made of a metallic material such as aluminum (Al) or a fluororesin material such as polytetrafluoroethylene (PTFE). The inner surface 21 of the housing 20 may be covered with the member described above. Alternatively, the housing 20 may be made of the member described above. For example, the ultraviolet reflectivity of the inner surface 21 is enhanced and the housing 20 can be used as a heat sink for the light emitting devices 40 by using aluminum to form the housing 20 and mirror finishing the inner surface 21.

The plurality of light emitting devices 40 are provided inside the housing 20 and outside the flow tube 12 and are arranged along the longitudinal direction of the flow tube 12. The light emitting devices 40 are light emitting diodes (LEDs) configured to emit ultraviolet light, and the central wavelength or peak wavelength thereof is included in a range of about 200 nm [symbol] 350 nm. It is preferable that the light emitting devices 40 be LEDs emitting ultraviolet light near a wavelength range of 260 nm [symbol] 270 nm having a high sterilizing efficiency. Such ultraviolet LEDs are exemplified by aluminum gallium nitride (AlGaN) based LEDs.

The plurality of light emitting device 40 are mounted on the substrate 42. The substrate 42 is mounted to one of the four wall surfaces forming the side wall 22. The substrate 42 is made by using a highly exoergic member. For example, copper (Cu), aluminum (Al), or the like is used as a base material.

The first spacer 34 is a ring-shaped member and accepts the flow tube 12 guided therethrough. The first spacer 34 is sandwiched between the first end wall 24 of the housing 20 and the first connector 30. Similarly, the second spacer 36 is a ring-shaped member and accepts the flow tube 12 guided therethrough. The second spacer 36 is sandwiched between the second end wall 26 of the housing 20 and the second connector 32. The first spacer 34 and the second spacer 36 are made of fluororesin such as PTFE.

The first spacer 34 and the second spacer 36 sandwich and fix the housing 20 between the first connector 30 and the second connector 32. Securing the housing 20 in this way eliminates the need for a structure to fix the housing 20 to the outer circumferential surface of the flow tube 12 and simplifies the structure of the sterilization device 10. The inventive structure also allows the first spacer 34 to clog a gap between the flow tube 12 and the first opening 25 and allows the second spacer 36 to clog a gap between the flow tube 12 and the second opening 27 so that the ultraviolet light emitted by the light emitting devices 40 is prevented from leaking outside the housing 20.

According to the structure described above, the fluid subject to the treatment by the sterilization device 10 flows into the flow tube 12 via the first connector 30, passes through the flow tube 12, and is discharged from the second connector 32. The fluid passing through the flow tube 12 is irradiated with the ultraviolet light from the plurality of light emitting devices 40 and is also irradiated with the ultraviolet light reflected by the inner surface 21 of the housing 20. In this process, the ultraviolet light from the light emitting devices 40 is reflected by the inner surface 21 formed by the four wall surfaces surrounding the flow tube 12 so that the ultraviolet light intensity is uniformized in the interior space of the irradiation chamber 50. This allows the fluid flowing through the flow tube 12 to be evenly irradiated with ultraviolet light.

According to the embodiment, the device is structured such that the fluid subject to the treatment passes inside the flow tube 12, the first connector 30, and the second connector 32 so that there is no need for a structure to seal between the flow path and the light emitting devices. If a plate member or a tubular member made of quartz glass is used for the purpose of transmitting ultraviolet light, a structure for sealing a gap between the member and the housing with, for example, an O-ring will be necessary with the result that the size of the device will be increased, and the number of components and cost will also be increased. Meanwhile, according to the embodiment, the light emitting devices 40 are arranged outside a simple flow path structure formed by the flow tube 12, the first connector 30, and the second connector 32 so that there is no need for a special sealing structure. Accordingly, a small and low-cost sterilizing device 10 is realized.

According to the embodiment, the outer circumference of the flow tube 12 is surrounded by the housing 20 that bounds the irradiation chamber 50 so that the fluid is efficiently irradiated with the ultraviolet light from the light emitting devices 40. Further, by configuring the housing 20 to have a quadrangular cross sectional shape, which is non-analogous to the circular cross sectional shape of the flow tube 12, reflection by the inner surface 21 of the housing 20 is exploited to uniformize the ultraviolet light inside the irradiation chamber 50. By uniformizing the intensity of ultraviolet light radiated, the fluid is prevented from being discharged without being sterilized sufficiently due to local shortage of the ultraviolet irradiation level. Thus, in accordance with the embodiment, the sterilization capability of the sterilization device 10 is improved with a simple structure.

According to the embodiment, a structure of sandwiching and fixing the housing 20 between the first connector 30 and the second connector 32 is used instead of a structure of fixing the housing 20 to the outer circumferential surface of the flow tube 12. Therefore, the structure to fix the housing 20 is simplified. The inventive structure also reduces the risk of a force being exerted to the outer circumference surface of the flow tube 12 and so reduces the likelihood of damage to the flow tube 12. Thus, according to the embodiment, the durability of the sterilization device 10 is enhanced.

According to the embodiment, the housing 20 is structured so as not be in contact with the fluid so that the inner surface 21, etc. of the housing 20 is prevented from being corroded by the fluid. In particular, where aluminum is used to form the inner surface 21 of the housing 20 and tap water is run as a fluid, chlorine and minerals contained in the tap water react with aluminum to deteriorate the inner surface. Meanwhile, according to the embodiment, the device is structured so as not to allow the fluid and aluminum to be in contact. Therefore, the inner surface 21 of the housing 20 is prevented from being corroded to result in lowered reflectivity and ultraviolet irradiation efficiency.

Described above is an explanation based on an exemplary embodiment. The embodiment is intended to be illustrative only and it will be obvious to those skilled in the art that various modifications to constituting elements and processes could be developed and that such modifications are also within the scope of the present invention.

In one variation, the cross section of the housing 20 may be shaped in a polygon such as a triangle and a hexagon. It is desired to avoid a pentagonal cross sectional shape of the housing. This is because, if a pentagonal housing is employed, the ultraviolet light reflected by the wall surfaces of the pentagon is concentrated in a limited portion so that the ultraviolet light intensity is prevented from being uniformized and the irradiation level becomes uneven.

In one variation, the inventive sterilization device may be used for a purification process that decomposes organic substance included in a fluid by using ultraviolet irradiation.

It should be understood that the invention is not limited to the above-described embodiment, but may be modified into various forms on the basis of the spirit of the invention. Additionally, the modifications are included in the scope of the invention.

What is claimed is:

1. A sterilization device comprising:
a flow tube that extends in a longitudinal direction;
a housing that extends in the longitudinal direction along the flow tube and surrounds an outer circumference of the flow tube; and
a light emitting device provided inside the housing and outside the flow tube and irradiating a fluid flowing in the flow tube with ultraviolet light, wherein
an inner surface of the housing is made of a material that reflects the ultraviolet light from the light emitting device, and
a shape of a cross section of the inner surface of the housing perpendicular to the longitudinal direction is non-analogous to a shape of a cross section of the flow tube perpendicular to the longitudinal direction.

2. The sterilization device according to claim 1, wherein
the shape of the cross section of the flow tube is circular and the shape of the cross section of the inner surface of the housing is polygonal.

3. The sterilization device according to claim 2, wherein
the shape of the cross section of the inner surface of the housing is quadrangular.

4. The sterilization device according to claim 1, wherein
the flow tube is made of a resin material that transmits the ultraviolet light from the light emitting device.

5. The sterilization device according to claim 1, further comprising:
a first connector connected to a first end of the flow tube; and
a second connector connected to a second end of the flow tube, wherein
the housing is sandwiched and fixed between the first connector and the second connector.

6. The sterilization device according to claim 5, wherein
the housing includes a side wall that extends along the longitudinal direction, a first end wall formed with a first opening through which the flow tube is guided, and a second end wall formed with a second opening through which the flow tube is guided, and
the sterilization device further comprises a first spacer provided between the first end wall and the first connector to surround the outer circumference of the flow tube, and a second spacer provided between the second end wall and the second connector to surround the outer circumference of the flow tube.

7. The sterilization device according to claim 5, wherein
an inflow port of the sterilization device is provided in the first connector and an outflow port of the sterilization device is provided in the second connector.

8. The sterilization device according to claim 1, wherein
the housing includes a plurality of side walls that extends along the longitudinal direction, and
the light emitting device is provided on an inner surface of only one of the plurality of side walls of the housing.

9. The sterilization device according to claim 8, wherein
a plurality of light emitting devices are arranged in the longitudinal direction on the inner surface of one of the plurality of side walls of the housing of the housing, and
the housing is made of aluminum (Al) and is configured as a heat sink for the plurality of light emitting devices and as a reflecting member that reflects ultraviolet light output from the plurality of light emitting devices.

10. The sterilization device according to claim 1, wherein
only one flow tube is provided inside the housing.
